(19) United States
(12) Patent Application Publication
(10) Pub. No.: US 2023/0263378 A1
(43) Pub. Date: Aug. 24, 2023

(54) APPARATUS, SYSTEMS AND METHODS FOR UNIFIED ENDOSCOPIC PROCEDURE PERFORMANCE AND VISUALIZATION

(71) Applicant: University of Iowa Research Foundation, Iowa City, IA (US)

(72) Inventors: Jarrett Walsh, MD, Iowa City, IA (US); Michael Puricelli, Iowa City, IA (US)

(21) Appl. No.: 18/014,067

(22) PCT Filed: Jul. 1, 2021
(86) PCT No.: PCT/US2021/040213

§ 371 Date: Dec. 30, 2022

Related U.S. Application Data
(60) Provisional application No. 63/046,740, filed on Jul. 1, 2020.

(51) Int. Cl. A61B 1/018; A61B 1/00; A61B 1/015

(52) U.S. Cl. CPC A61B 1/018; A61B 1/015; A61B 1/00147

(57) ABSTRACT

The disclosed apparatus, systems and methods relate to an endoscopic devices configured to house a telescope and having a rotating tube configured for the application of suction or laser excision. A flexion cartridge can be provided that allows for the flexion of a laser for positioning in the surgical theater. The rotating tube is configured to rotate the laser around the scope tube.

### CROSS-REFERENCE TO RELATED APPLICATIONS

This application claims priority to U.S. Provisional Application No. 63/046,740 filed Jul. 1, 2020 and entitled “Apparatus, Systems And Methods For Unified Endoscopic Procedure Performance And Visualization,” which is hereby incorporated by reference in its entirety.

### TECHNICAL FIELD

The present disclosure relates to devices for certain surgical procedures, such as endoscopy.

### BACKGROUND

The disclosure relates to apparatus, systems and methods for use in endoscopic procedures.

Removal of airway foreign bodies and treatment of certain pathologies is aided by the use of endoscopic instrumentation. Prior art instrumentation has limitations in certain procedures. There is a need in the art for improved interventional delivery and methods that can be used under simultaneous endoscopic visualization.

### BRIEF SUMMARY

Discussed herein are various devices, systems and methods relating to endoscopic and otolaryngology (“ENT”) procedures including laryngoscopy and bronchoscopy as well as other procedures wherein a guided endoscopic delivery is necessary, such as aesthetic dermatology and plastic surgery, podiatry, gynecology, neurosurgery, orthopedics, general and thoracic surgery (including open and endoscopic), dental and oral surgery, and genitourinary surgery. Certain non-limiting examples include epistaxis/hereditary hemorrhagic telangiectasia with a shorter scope, middle ear and mastoid surgery, colposcopy with intervention, and potentially many others in different disciplines. otology myringoplasty, tympanoplasty, ossicular surgery, stapes surgery, cholesteatoma excision, chronic otitis media, laryngology vocal nodule ablation, subglottic stenosis incision/excision, tracheal stenosis incision/excision suprastomal collapse excision, neoplasm excision, supraglottoplasty, nasal neoplasm excision, epistaxis/vascular cauterization, head and neck ablative procedures; urology such as for ureteral stones, benign prostatic hyperplasia, and others; colorectal applications including lesion ablation, as well as laparoscopic procedures and thoracic procedures.

In Example 1, an endoscopic device comprising an elongate body comprising: a rotating tube comprising a distal rotating tube end and a proximal rotating tube end; and a scope tube, the scope tube comprising a distal scope tube end and a proximal scope tube end, wherein the rotating tube is configured for the introduction of a surgical tool and / or suction to the distal rotating tube end for colocalization with a telescope introduced through the scope tube.

In Example 2, the device of Example 1, wherein the rotating tube is configured to accommodate a laser.

In Example 3, the device of Example 2, wherein the rotating tube is in rotational communication with the scope tube.

In Example 4, the device of Example 2, wherein the rotating tube comprises a flexion cartridge disposed therein.

In Example 5, the device of Example 4, wherein the flexion cartridge comprises a plurality of flexion units configured to articulate the cartridge.

In Example 6, the device of Example 4, wherein the flexion cartridge is configured to be capable of advancement beyond the distal end of the rotating tube.

In Example 7, wherein the rotating tube is configured to provide suction.

In Example 8, an endoscopic device comprising a rotating tube defined in the lumen, the rotating tube comprising a distal rotating tube end and a proximal rotating tube end, a scope tube, the scope tube comprising a distal scope tube end and a proximal scope tube end; and a flexion cartridge disposed within the rotating tube.

In Example 9, the device of Example 8, wherein the rotating tube is in rotational communication with the scope tube.

In Example 10, the device of Example 8, wherein the flexion cartridge comprises a plurality of triangular flexion units configured to articulate the cartridge.

In Example 11, the device of Example 8, wherein the flexion cartridge is configured to be capable of advancement beyond the distal end of the rotating tube.

In Example 12, the device of Example 8, wherein the flexion cartridge is in operational communication with at least one actuator button.

In Example 13, the device of Example 8, wherein the flexion cartridge is in operational communication with at least one cable.

In Example 14, the device of Example 8, further comprising a locking mechanism.

In Example 15, an endoscopic device comprising a substantially rigid sheath comprising a lumen and having a distal sheath end and proximal sheath end, a rotating tube defined in the lumen, the rotating tube comprising a distal rotating tube end and a proximal rotating tube end; a scope tube defined in the elongate tube opposite the rotating tube, the scope tube comprising a distal scope tube end and a proximal scope tube end; a semi-rigid flexion cartridge disposed at the rotating tube distal end; and at least one locking mechanism.

In Example 16, the endoscopic device of Example 15, wherein the rotating tube is configured to rotate around the scope tube.

In Example 17, the endoscopic device of Example 15, wherein the flexion cartridge is configured to extend beyond the distal end of the scope tube.

In Example 18, the endoscopic device of Example 15, wherein the flexion cartridge is configured to extend beyond the distal end of the rotating tube.

In Example 19, the endoscopic device of Example 15, wherein the flexion cartridge comprises a laser lumen.

In Example 20, the endoscopic device of Example 15, wherein the flexion cartridge comprises a fluidic lumen.

In Example 21, the endoscopic device of Example 15, further comprising at least one actuation button configured to advance the flexion cartridge distally beyond the distal end of the scope tube.

In Example 22, the endoscopic device of Example 21, wherein the flexion cartridge is advanced by nudge.

In Example 23, the endoscopic device of Example 21, wherein the flexion cartridge is advanced by slide.

In Example 24, the endoscopic device of Example 15, wherein the rotating tube is configured to house and rotate a tool in the surgical theater.

In Example 25, the endoscopic device of Example 24, wherein the tool is selected from the group consisting of a plasma tool, a coblation tool, a radiofrequency tool, a monopolar cautery tool, a bipolar cautery tool and a needle.

In Example 26, the endoscopic device of Example 15, wherein the flexion cartridge comprises a laser lumen.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A is a perspective view of the device, according to an exemplary embodiment.

FIG. 1B is an proximal endlong view of the device, according to an exemplary embodiment.

FIG. 1C is a proximal perspective view of the device, according to an exemplary embodiment.

FIG. 2A is a side view of the device, according to an exemplary embodiment.

FIG. 2B is a side view of the device of FIG. 2A, including a scope.

FIG. 3A is a side view of the device, according to an exemplary embodiment.

FIG. 3B is a perspective view of the device of FIG. 3A.

FIG. 3C is a cutaway view of the implementation of FIG. 3A.

FIG. 3D is a distal endlong view of the device, according to the implementation of FIG. 3A.

FIG. 3E is a proximal endlong view of the device, according to the implementation of FIG. 3A.

FIG. 3F is a closeup view of the proximal, handle region of the implementation of FIG. 3A.

FIG. 4A is a side view of the device, according to an exemplary embodiment.

FIG. 4B is a side view of the device of FIG. 2A, including a scope.

FIG. 4C is a side view of an angled laser for insertion into the rotating tube, according to one implementation.

FIG. 5A is a side view schematic of the distal end of the device, according to one implementation.

FIG. 5B is a side view schematic of the distal end of the device, according to one implementation.

FIG. 5C is a side view schematic of the distal end of the device, according to one implementation.

FIG. 6A is a side view of the device, according to one exemplary implementation .

FIG. 6B is a cutaway side view of the device, according to one exemplary implementation.

FIG. 6C is an underside view of the device, according to one exemplary implementation.

FIG. 6D is an topside view of the device, according to one exemplary implementation.

FIG. 7A is a perspective view of the device, according to one implementation.

FIG. 7B is a close up perspective view of the distal end of the device of FIG. 7A.

FIG. 7C is a close up endlong view of the distal end of the device of FIG. 7A.

FIG. 8A is a close up perspective view of one implementation of the device having a scope.

FIG. 8B is a close up perspective view of the implementation of FIG. 8A with the scope removed.

FIG. 9A is a close up side view of the distal end of the device according to one implementation, wherein the flexion cartridge is in a retracted position.

FIG. 9B is a close up side view of the distal end of the device according to one implementation, wherein the flexion cartridge is in an advanced nudge position.

FIG. 9C is a close up side view of the distal end of the device according to one implementation, wherein the flexion cartridge is in an advanced nudge and rotated position.

FIG. 9D is a distal endlong view of the device having the rotating tube in a first position, according to one implementation.

FIG. 9E is a distal endlong view of the device having the rotating tube in a second position, according to one implementation.

FIG. 10A is a side view of the device according to one implementation wherein the rotating tube and cartridge are in the retracted slide position.

FIG. 10B is a side view of the device according to the implementation of FIG. 10A wherein the rotating tube and cartridge are in the advanced slide position.

FIG. 10C is a side view of the device according to another implementation wherein the rotating tube and cartridge are in the retracted slide position.

FIG. 10D is a side view of the device according to the implementation of FIG. 10C wherein the rotating tube and cartridge are in the advanced slide position.

FIG. 10E is a side view of the device according to one implementation wherein the rotating tube and cartridge are in the retracted slide position.

FIG. 10F is a side view of the device according to the implementation of FIG. 10E wherein the rotating tube and cartridge are in the advanced slide position.

FIG. 11A is a close up side view of the distal end of the device according to one implementation, wherein the flexion cartridge is in a retracted nudge position.

FIG. 11B is a close up side view of the distal end of the device according to one implementation, wherein the flexion cartridge is in an advanced nudge position.

FIG. 11C is a close up side view of the distal end of the device according to one implementation, wherein the flexion cartridge is in an advanced nudge and flexion position.

FIG. 12 is a distal endlong view of the device according to one implementation.

FIG. 13A is a close up side view of the distal end of the device according to one implementation, wherein the flexion cartridge is in an extension position.

FIG. 13B is a close up side view of the distal end of the device according to one implementation, wherein the flexion cartridge is in an flexion position.

### DETAILED DESCRIPTION

The various embodiments disclosed or contemplated herein relate to an endoscopic device for use in certain medical procedures such as laryngoscopy and bronchoscopy, as well as certain implementations utilizing optical laser fiber introduction in laryngoscopy and bronchoscopy as well as otolaryngology, gynecology, urology, colorectal surgery and the like.

In various implementations, the disclosed device allows for the co-introduction of several surgical tools via a single device comprising several features, so as to allow for the surgeon or user to position the distal ends of the tools in the surgical theater via a single introductory device, such that the tools introduced via the introductory device can be positioned and manipulated independently, and suction can be applied, as would be understood from the foregoing description.

It is understood that in certain procedures, grasping a foreign object may not be as beneficial as applying directed suction. Currently, the only approach to applying suction at a distance when using rigid bronchoscopy is through the use of a fine suction catheter, fed into the rotating tube of the bronchoscope along with the optical instrumentation. However, the diameter of the rotating tube on the bronchoscopes are too small to pass a suction of meaningful size. Another option is to place a larger diameter suction tool without direct visualization, which can traumatize surrounding tissue or displace the object deeper, thus compromising the removal of the foreign object.

The disclosed devices, systems and methods allow direct visualization and interaction with the surgical theater by one or more tools for precise placement while providing adequate suction to interact with and resect tissue or remove foreign bodies, apply thermal energy to interact with tissue in a clinically desired manner for procedures such as excision, ablation, coagulation and the like. That is, the disclosure relates to the introduction and manipulation of surgical tools to a surgical theater for use via a single, unified delivery device.

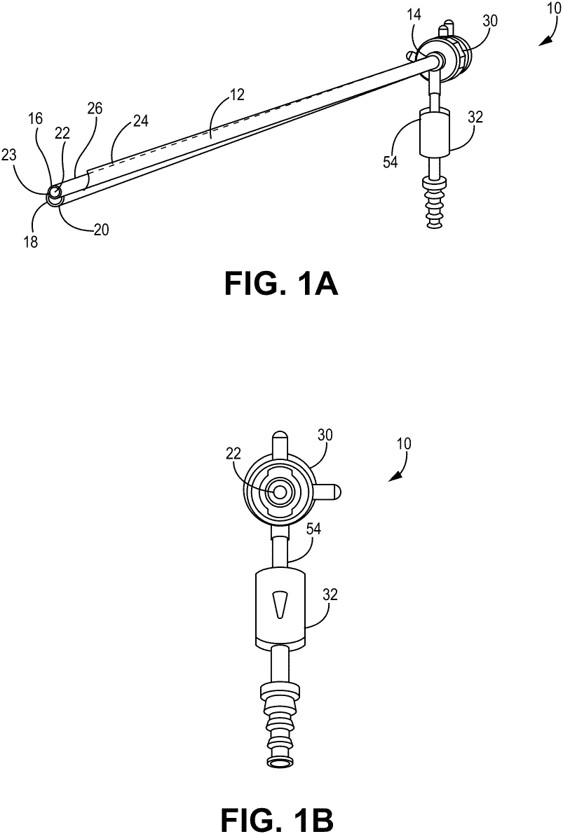

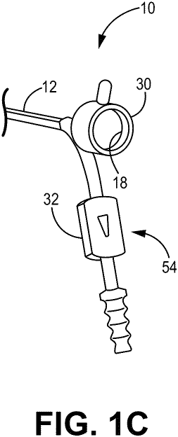

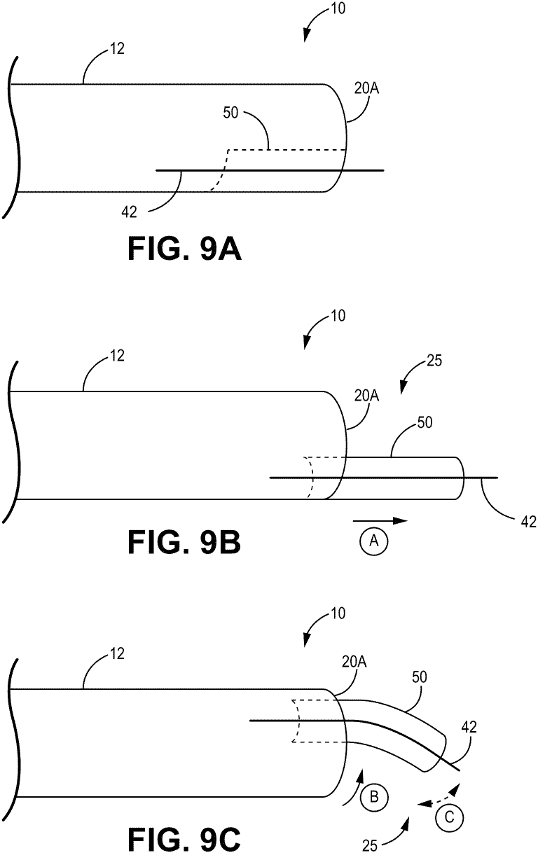

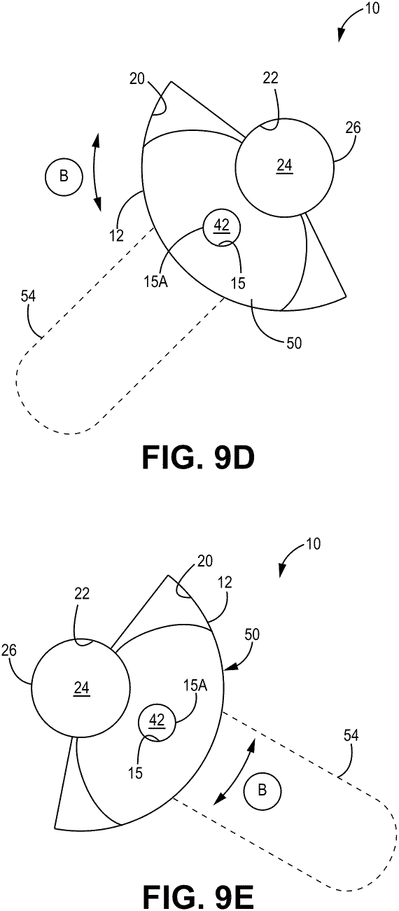

Turning to the figures in greater detail, FIGS. 1A-1C depict perspective and cross-sectional views of the device 10 according to certain implementations. In these implementations, the device 10 comprises an elongate body 12 having a proximal end 14 and a distal end 16 and defining at least one lumen 18. The elongate body 12 is made up of two separate structures: an elongate rotatable tube 20 and a central scope tube 24 disposed adjacent to or in contact with the rotatable tube 20 such that the rotatable tube 20 is moveable in relation to the scope tube 24 as discussed in further detail below. In certain implementations, the elongate body 12 is rigid from the proximal end 14 to the distal end 16, while in alternate implementations, a semi-rigid distal end portion 50, as is shown in FIG. 1D and below FIGS. 9A-13B.

Continuing with FIGS. 1A-1C, in certain implementations, the rotatable tube 20 has a lumen 18 and the scope tube 24 has a lumen 23 defined therein that is adapted to house a telescope 26, as described herein.

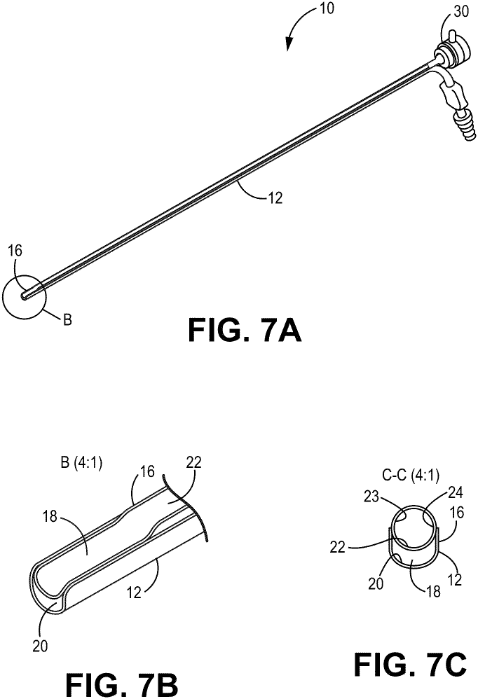

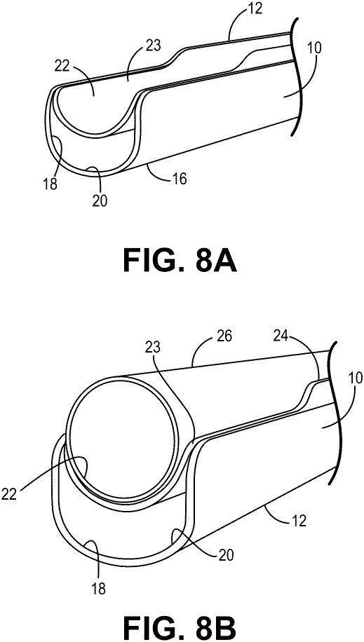

According to certain embodiments, the scope tube 24 can be circular or semi-circular in shape, and the rotatable tube 20 can be rotated relative to the scope tube 24. More specifically, as best shown in FIG. 8B, in various implementations, the distal end of the scope tube 24 is disposed adjacent to the rotatable tube 20 such that the scope tube 24 is central to the surgical theater relative to the position of the rotatable tube 20. That is, as best shown in FIGS. 7B-8B, the distal end of the rotatable tube 20 has a cross-sectional profile similar to a half moon such that a channel 22 is defined in the wall of the rotatable tube 20 as shown. More specifically, the cross-section of the channel 22 wall has a circumference of less than 360°. In most embodiments, the circumference of the channel 22 wall is about 180°. Alternatively, the circumference of the channel 22 wall can range from about 100° to about 300°.

As such, the distal end of the scope tube 24 is disposed within the channel 22 defined in the rotatable tube 20 such that the rotatable tube 20 can rotate around the centrally-disposed scope tube 24 and scope (not shown), as shown in the disclosed implementations. Further, in accordance with certain implementations, the distal scope tube 24 can also have a wall circumference that is less than 360° (or any circumference in any of the ranges described above with respect to the channel 22) with the gap defined in the cross-section of the tube 24 disposed in a complementary fashion with the channel 22 such that the lumen of the scope tube 24 is defined in part by the wall of the scope tube 24 and in part by the wall of the channel 22. It is further understood that the scope tube 24 according to certain implementations is present in the proximal region but may not extend the full length of the body 12, such that the channel 22 is responsible for supporting the scope at the distal end 16.

Further, as best shown in FIG. 7A, the channel 22 defined by the wall of the rotatable tube 20 extends along a length of the rotatable tube 20 such that the scope tube 24 is disposed within the channel 22 along almost the entire length of the body 12. However, along a length of the rotatable tube 20 near the proximal end of the tube 20, the wall of the channel 22 extends entirely around the outer circumference of the scope tube 24 such that the channel 22 becomes a lumen with the scope tube 24 disposed therein. That is, at some point along the length of the rotatable tube 20 near the proximal end, the wall of the channel 22 has a circumference of 360° such that the scope tube 24 is fully enclosed within the channel 22.

In implementations like those of FIGS. 1A-1C, the rotatable tube 20 is constructed and arranged to allow for the introduction of surgical tools such as lasers or other manipulable tools, and / or provide suction through the lumen 18 of the rotatable tube 20 for use in various procedures, such as the extraction of foreign bodies. In various implementations, and as shown for example in FIGS. 1A-1C, the device 10 comprises an optional suction port 32 for the provision of suction, such as on the handle 54, which is in operational communication with the device 10 and rotating tube 20 as described herein.

It is understood that in certain of these implementations, the lumen 18 of the rotatable tube 20 is sized to sufficiently allow fluidic communication of suction from the suction port 32 to the distal end 16 of the device 10 for use in a variety of procedures, such as extraction of foreign bodies and aspirated food or liquid. In various implementations, the suction provided to the distal end via the elongate body 12 and rotatable tube 20 exceeds that which has been possible in prior art flexible devices. For example, in certain implementations the suction provided is about 800 mmHg or more which is approximately ten times that of the largest known diameter suction catheter in use and twenty times that of the smallest known diameter suction catheter in use.

In initial prototype testing, the mass lifted via the rotatable tube 20 was assessed, and the results are shown in Table 1:

TABLE 1

Mass Lifted Via Suction

| Orifice | Mass Lifted (grams) |
|---|---|
| 5 French | 2.0 |
| 7 French | 4.0 |
| 3 Holes Open on Prototype | 2.0 |
| 2 Holes Open on Prototype | 3.5 |
| 1 Hole Open on Prototype | 12.0 |
| All Holes Closed | 45.0 |

It is readily appreciated that a variety of suction strengths can be provided sufficient to perform the various tasks described herein. In alternate implementations, and as described below, passage for the removal of smoke or other gases is provided passively, and without suction.

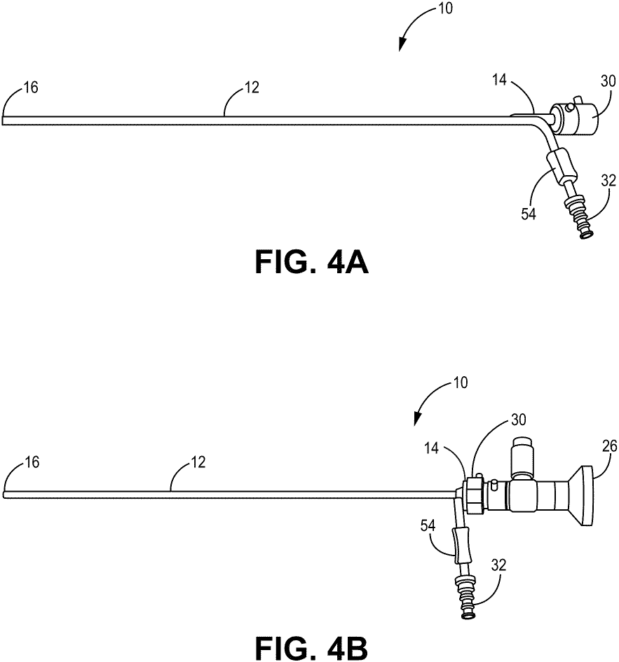

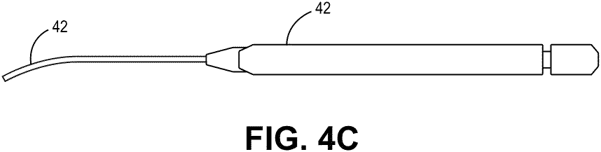

In various implementations, the rotatable tube 20 is adapted to provide a laser through the lumen 18 for use in the excision, ablation, coagulation or other treatment of pathologies. For example, in certain implementations the rotatable tube 20 is adapted to provide a CO2 laser (shown, for example, in FIG. 4C at 42) through the lumen 18 to the distal end 16 of the device for use in procedures such as radial incision of subglottic stenosis, excision of laryngeal tumors, ablation of dysplastic lesions, ablation of mastoid tissue such as cholesteatoma, and coagulation of recurrent epistaxis. Further uses would be readily apparent to the skilled physician.

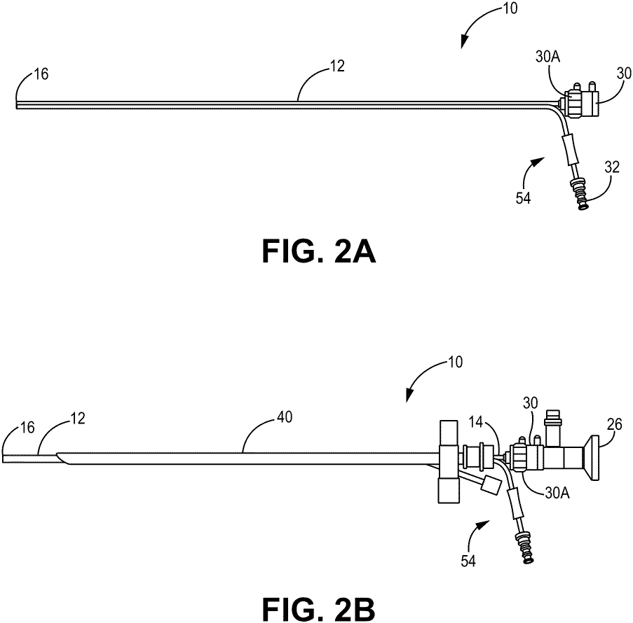

As shown in FIGS. 1B-1C and in FIGS. 2A-B, in various implementations, the telescope port 30 and scope tube 24 are sized to accommodate a telescope 26, such as a telescope having an elongate tube (not shown) that is about 1.9 mm, about 2.7 mm or about 4 mm in diameter, though other diameters are of course possible and would be readily appreciated by those of skill in the art. In addition, the length of the instrument shown accommodates a telescope approximately 36 cm long. Other iterations would accommodate a range of other lengths for various clinical applications and would be readily appreciated by those of skill in the art.

The scope tube 24 according to certain implementations is also configured to allow for the visualization of the surgical theater in several angles, such as being sized and shaped such that the opening defined in the distal end 24A of the scope tube 24 is sized and shaped to allow for the movement or angling of the distal end of the telescope 26 to move or angle, as would be appreciated.

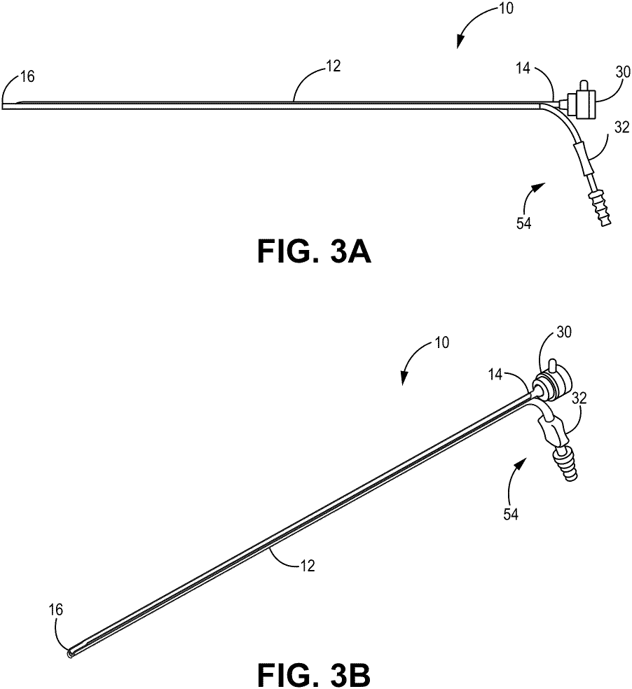

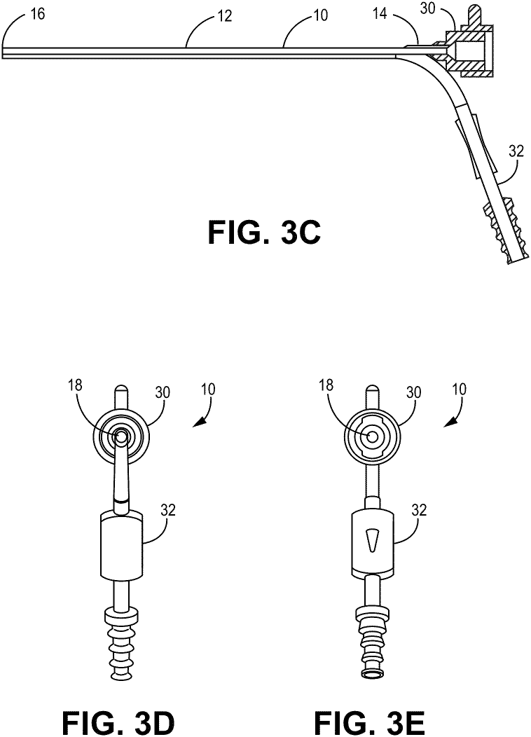

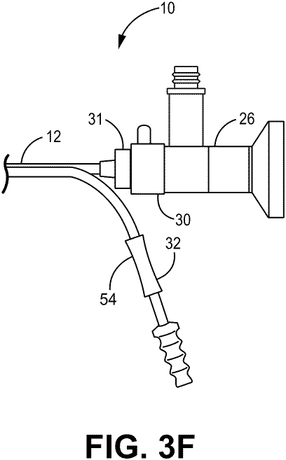

As shown in FIGS. 1A-3B, the device 10 comprises a telescope port 30 comprising an external ring 30 configured to articulate, optionally with a common endoscopic bridge adapter 30A (shown in FIGS. 3A-3B), to allow for tight articulation of the telescope 26 relative to the device 10 and easy transition of the telescope 26 from a rigid bronchoscope to the device 10, as would be readily appreciated. In various implementations, and as shown in FIGS. 2C and 3A-3F, the endoscopic bridge 30A is not required and the articulation and locking functions of a typical bridge are achieved by the device 10. In these implementations, the external ring 30 is a rotating lock (¼ rotation) to secure the telescope 26 into place. That lock mechanism is attached to and is part of the internal portion as a single working mechanism, as shown in FIG. 3C at 30.

Further, a rotation tube lock 31 is also provided in certain implementations, which is constructed and arranged to lock the circumferential position of the rotating tube 20 relative to the elongate body 12 / scope tube 24, as would be appreciated.

As shown in the implementations of FIGS. 2B and 3F, the device 10 and elongate body 12 are constructed and arranged to be placed inside a ventilating bronchoscope 40 for use, as would be readily appreciated. In these implementations, the ventilating bronchoscope 40 can be used to provide ventilation to the patient while a procedure is occurring via the device 10, as would be understood.

In use according to the implementations of FIGS. 2B-3F, the device 10 is introduced via the ventilating bronchoscope 40 such that the telescope 26 and rotatable tube 20 can be used in a procedure while the patient is also ventilated. That is, in use according to certain implementations, the device 10 is used to observe and locate, for example, a foreign body or pathology via the telescope 26 for application of suction or laser excision (as discussed in relation to FIG. 4C) through the rotatable tube 20, as would be readily appreciated. Such use improves upon the current state of the art in that it allows for visualization of the working theater and application of treatment via the same instrument and therefore avoids the necessity of introducing separate instruments which can be cumbersome and less effective or, by their size, preclude the use of a ventilating bronchoscope.

In certain implementations, the device 10 can also be utilized without a bronchoscope sheath for direct manipulation and visualization of the upper airway or other locations outside of the trachea and bronchi. In certain instances, the instrument may be placed through a trochar or other introducer to be used in various body cavities.

FIGS. 3F and 4A-4B depict implementations of the device 10 adapted to accommodate a telescope 26. In certain implementations, the elongate body 12 is constructed and arranged for articulation or being posed at a variety of angles. In these implementations, the elongate body 12 or sheath 12 has at least one point wherein the axial direction shifts such that the openings at the proximal end 14 and distal end 16 are no longer coplanar and the specialist can navigate the distal end 16 around a curved or cornered bodily passageway whilst still maintaining direct visualization.

In various implementations, a laser fiber 42 can be inserted into the rotating tube shown elsewhere at 20. Such laser fiber 42 placement offers significant advantages over microscope mounted laser delivery and flexible fiber delivery.

Several challenges are presented by current laser usage. It is appreciated that non-fiber microscope mounted laser use is associated with proximal tissue burns and an inability to direct the fiber around focal narrowing, such as the vocal folds prohibiting access to the trachea or nostril limiting access to the nasal cavity. Further, due to the narrow and long access for many procedures, including those through natural body orifices, near parallel placement is required, and contact between the separate telescope and fiber complicate delivery.

Flexible endoscopic laser delivery is utilized in some cases, but flexible delivery permits less precise distal instrument control. It is further understood that the rotating tubes of prior art endoscopes can be too narrow, or otherwise incompatible with flexible laser delivery systems. These prior art systems also present a risk of a flexible laser fiber bending at too extreme of an angle to function.

Therefore, the ability of certain device 10 implementations to pass a laser fiber 42 carrying instruments with an adjacent telescope 26 to provide endoscopic visualization presents a significant advantage over the prior art, in that, for example, it allows the user to have multiple tools introduced via a single device which allows them to avoid cumbersome introduction of multiple devices through narrow openings or choke points, as well as for the co-localization of a tool with the scope for more precision in visualization and manipulation.

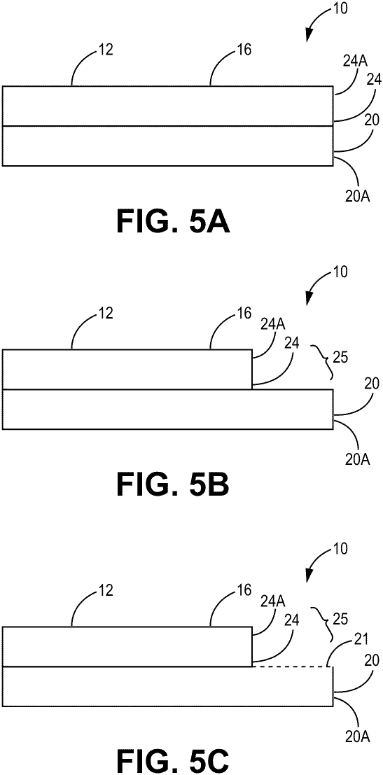

As shown in FIGS. 5A-5C, the distal end 16 can have several different configurations in certain embodiments. For example, as shown in FIG. 5A, the rotatable tube 20 and scope tube 24 of certain embodiments are co-terminal such that the distal end 24A opening of the scope tube 24 and the distal end 20A opening of the rotatable tube 20 are substantially aligned. It is appreciated that in all of these implementations, the distal ends 20A, 24A are open for view and use, as is best shown in the implementation of FIG. 2A.

In various alternate implementations like those of FIGS. 5B-5C, the distal end 24A of the scope tube 24 is offset proximally from the distal end 20A of the rotatable tube 20, so as to allow a working space 25 between the distal ends 20A, 24A of the tubes 20, 24 that can provide for suction or, as described later, operation of the laser or other energy-delivery tools or other tools described below.

In certain implementations, and as shown in FIG. 5C, a working portion 21 is provided near the distal end 20A of the rotatable tube 20. In certain of these implementations, the working portion 21 is fenestrated to allow for the application of, for example, suction laterally in the working space A, while in various alternate implementations a velvet eye 21 is utilized, as would be appreciated by those of skill in the art. In yet further implementations, the rotatable tube 20 comprises a laser lumen 15 configured to secure the laser 42 or other tool within the rotatable tube 20, such as at both the proximal and distal ends 20A, as would be readily appreciated and as described further below in relation to FIGS. 9D-9E. Further configurations are of course possible.

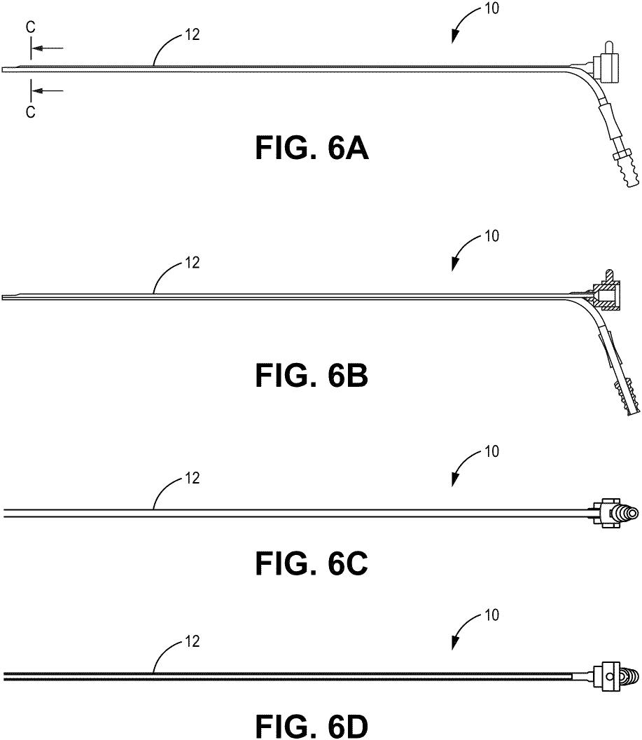

FIGS. 6A-6D depict several views of the device 10 according to one implementation. As shown in FIGS. 7A-7C, in these implementations, the device 10 comprises an open lumen 18 at the distal end 16, such that the rotatable tube 20 is enclosed while the scope tube 24 is open, as would be readily appreciated. FIGS. 8A-8B depict further views of the distal end 16 of the device 10 according to these implementations, with FIG. 8B depicting the open scope tube 24 housing the telescope 26 according to these implementations.

FIGS. 9A-13B depict implementations of the device 10 comprising a flexible, extendable semi-rigid distal end or flexion cartridge 50 is capable of being advanced beyond the distal end 16 of the scope 26 / scope tube 24, as described in detail below. That is, in various implementations, the flexion cartridge 50 is configured for being advanced and retracted via several approaches such as slide and nudge, as described herein. In in these implementations, the distal end of the rotating tube comprises the flexion cartridge: in some implementations the flexion cartridge is disposed within the rotating tube and is configured to be nudged distally out of the distal end of the rotating tube, while in alternate implementations the distal end of the rotating tube 18 is integrated with the cartridge and the cartridge is exposed for flexion upon the distal slide of the rotating tube, as would be appreciated.

That is, as shown in the implementations of FIGS. 9A-9C and elsewhere, it is appreciated that the distal flexion cartridge 50 is configured to be selectively advanced beyond the distal end 16 of the shaft into the working space 25 so as to allow for manipulation of a device such as a laser in the surgical theater, and later retracted for withdrawal, as described further herein and as would be readily appreciated. In certain implementations, the flexion cartridge 50 extends the length of the rotatable tube 20, while in other implementations, the cartridge 50 is inserted into the rotating tube 20 and disposed at the distal end 16 of the device for nudging or sliding, as described herein.

As also shown in FIGS. 9D-9E, in various implementations the device 10 is configured such that the rotatable tube 20 comprising the cartridge 50 can be rotated relative to the telescope 26 for circumferential positioning of the laser 42 or other tool in the surgical theater relative to the field of view of the user, which remains constant relative to the movement of the cartridge 50 as would be understood. In use according to these implementations, the circumferential or rotational position of the rotatable tube 20 / cartridge 50 is then locked, as described above in relation to FIG. 3F. It is appreciated that in these implementations, the telescope 26 is held in a constant position in the scope tube 24, which can be a depression in opposite the rotatable tube 20.

It is further understood that in certain of these implementations, the cartridge 50 defines a laser lumen 15 that secures the laser 42 or other introduced tool in place within the cartridge, such that movement of the cartridge 50 correspondingly repositions the laser 42 or other tool in the theater. In various of these implementations, the laser lumen 15 comprises a distal flange 15A configured to support and secure the laser 42 in place and, in the event of a break in the laser fiber, retain the laser fiber within the rotating tube so as not to enter the body of the patient.

In various implementations, other tools 42 can be introduced, certain non-limiting examples including plasma tools, coblation tools, radiofrequency tools, monopolar cautery tools and bipolar cautery tools, as well as administration devices such as needles or other devices for the injection or application of compounds, pharmaceuticals, topical application and the like, and others that would be readily appreciated in the art.

In certain implementations, the distal flange 15A is structurally integrated into the laser lumen 15, while in alternate implementations it is in a V-shape or other configuration constructed and arranged to support the laser 42 fiber during articulation and use and prevent breakage and the introduction of foreign bodies into the surgical theater. In alternate implementations, the flange 15A is provided as an alternative to the laser lumen 15, which is not presented. Further, in certain implementations, the laser fiber 42 is also secured at the proximal end of the rotatable tube 20 or device 10, again, such that in the event of a break in the laser fiber, it does not escape from the device and into the body of the patient, as would be appreciated.

While several implementations of such advanced positioning (e.g., nudge or slide) of the flexion cartridge 50 are disclosed herein, it would be readily apparent to those of skill in the art that further implementations are of course possible.

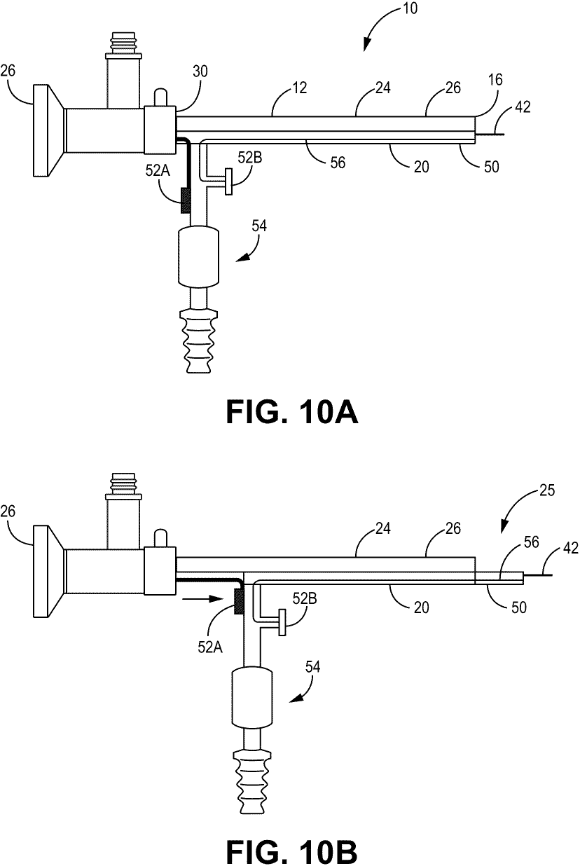

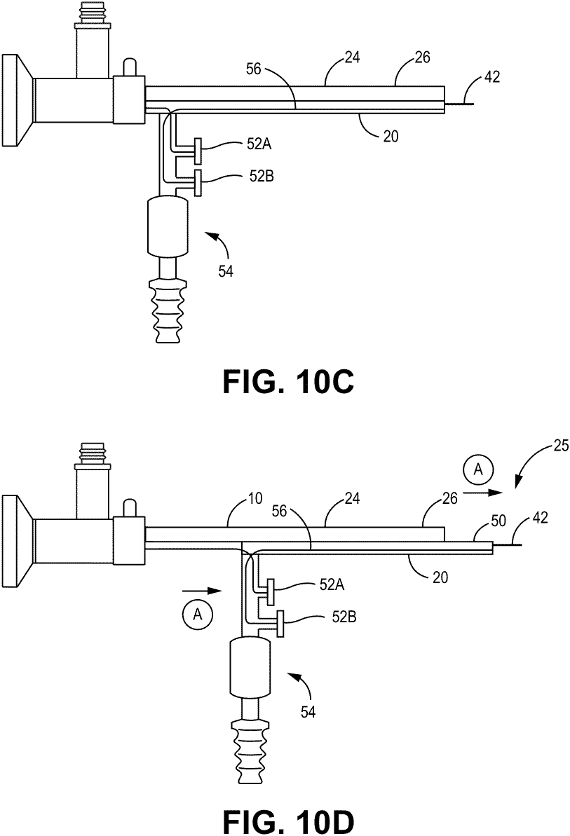

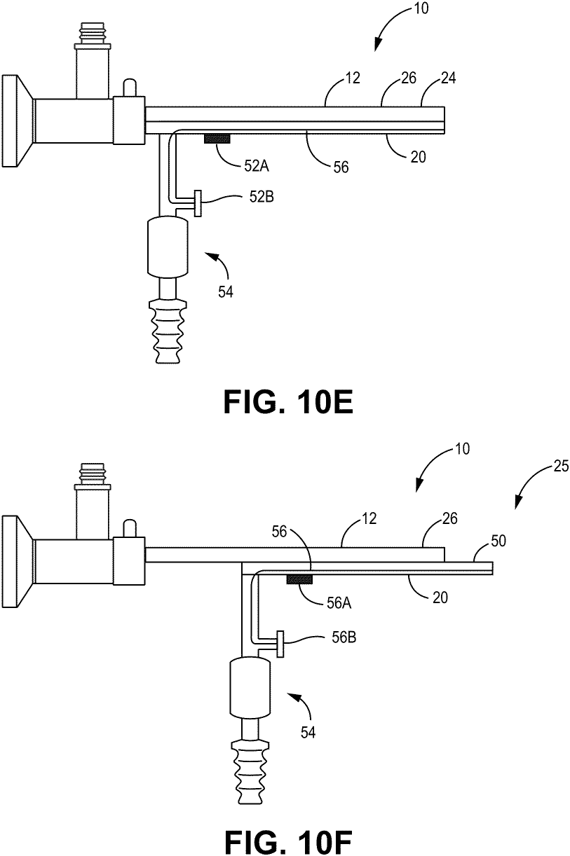

As shown in the implementations of FIGS. 10A-10F, in these various implementations, the devices 10 further comprise a handle 54 region that may comprise one or more actuation buttons 52 or other manipulable toggles or slides, as well as, in certain implementations, finger rests 56 for the user. In various implementations, the handle 54 and buttons 52 are in mechanical, positionable communication with the cartridge 50, and for example, a laser fiber 42, so as to allow for the extension of the laser fiber into the surgical theater, as well as the rotation of the laser relative to the scope and finally, the flexion and retraction of the flexion cartridge 50 so as to further position the laser in the surgical theater. That is, in various implementations, the flexion cartridge 50 is configured to be capable of three degrees of freedom: 1) being advanced/retracted, 2) rotation and 3) flexion, which can be understood as slide/nudge (reference arrow A), circumferential roll (reference arrow B) and 3) pitch/yaw (reference arrow C), as is shown for example in FIGS. 9A-9C.

As shown in FIGS. 10A-10B, 10C-10D and 10E-10F, several possible implementations can be used to effectuate the slide movement of the rotating tube 20, flexion cartridge 50 and, correspondingly, the accompanying device, such as a laser 42. It is appreciated that in certain of these implementations, the distal end of the laser 42 can be co-terminal with the distal end of the proximal end of the rotating tube 20 or can extend beyond the distal end of the rotating tube 20 in the retracted position, and be urged further into the surgical theater in the advanced position.

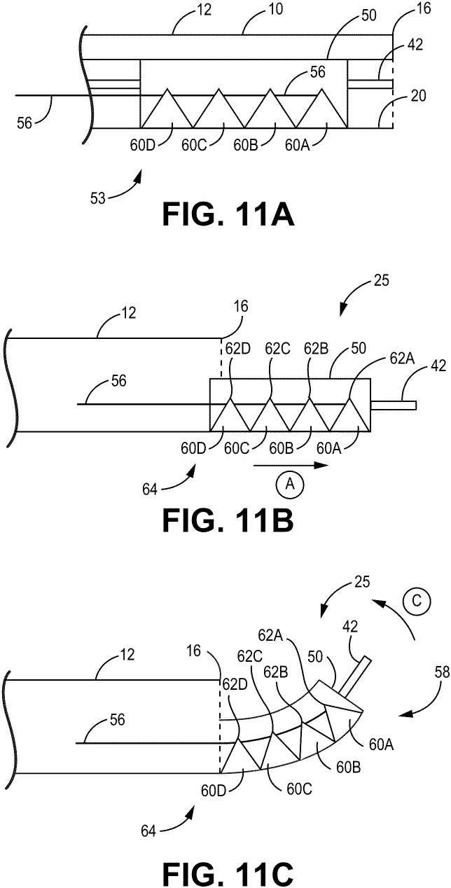

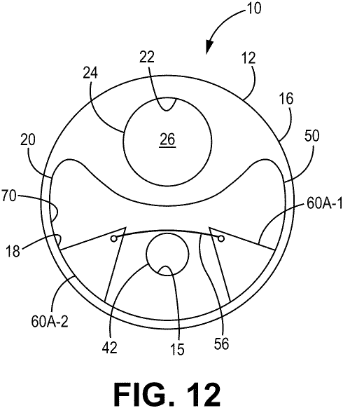

While these examples provide certain illustrative examples, it should be readily apparent to those of skill in the art that further examples are of course possible, wherein the various cables, buttons and similar components can be configured to effectuate the desired movement of the flexion cartridge 50. In addition to the slide configurations, in certain alternate implementations, the cartridge 50 is nudged beyond the distal end of the rotating tube 20 and scope tube 24, shown in FIGS. 9A-9E and 11A-11C or the slide configurations shown in FIGS. 10A-F. In either of these implementations, the actuation cable 56 or other system for actuating flexion is configured to extend with the cartridge 50 for later actuation / flexion, as would be readily appreciated.

As is shown in the slide implementations of FIGS. 10A-10F, the semi-rigid distal flexion cartridge 50 is capable of being advanced beyond the distal end 16 of the elongate shaft 12 through the rotating tube, such as via actuation of one or more manipulables 52 such as buttons 52 or other toggles or slides 52 disposed on an optional handle 54 provided in these implementations. In various implementations, the position of the handle 54 relative to the flexion cartridge 50 remains constant, but the handle 54 is urged distally or proximally by the user relative to the device 10 so as to adjust the extension of the flexion cartridge 50. It is appreciated that in certain implementations, the relative distance between the handle and distal end of the flexion cartridge 50 remains fixed, while in alternate implementations, the distance varies.

In the implementations of FIGS. 10A-10B, the handle 54 is in direct mechanical communication with the cartridge 50 and is in slidable communication with the device 10, such that urging the handle 54 (such as via a slide push 52A) distally results in a corresponding distal extension of the flexion cartridge 50, shown at reference arrow A. In these implementations, actuation of the roll is similarly achieved via the rotation of the handle about the axis of the scope.

For the third degree of freedom, flexion / extension is actuated via an flexion button 52B in operational communication with a cable 56 such that actuation of the flexion button 52B causes a corresponding retraction of the cable 56 from the flexion cartridge 50, resulting in the flexion articulation of the flexion cartridge 50, described in relation to FIGS. 11A-13B. Release of the flexion button 52B correspondingly results in a return extension of the cartridge 50, back to the linear position, as would be understood. It is appreciated that in the implementations of FIGS. 9A-9B, the flexion cartridge 50 is capable of three-degrees of freedom for use in the surgical theater in conjunction with the telescope 26 and any other tools introduced into the theater.

As shown in the implementations of FIGS. 10C-10D, a slide button 52A rather than a slide, is provided on the handle 54, such that a depression of the slide button 52A results in the urging of the cartridge 50 distally, into the theater, as would be appreciated.

In the implementation of FIGS. 10E-10F, the slide push 52A is disposed on the distal side of the handle 54 and is in operational communication with the cartridge 50 to slide advance the cartridge 50, as would be readily appreciated.

It is appreciated that in certain implementations, the distal end of the rotating tube 20 is semi-rigid or has a working space 25, such that the cartridge 50 is integrated into the distal end of the rotating tube 20 capable of flexion in the advanced position, that is, when the distal end of the rotating tube 20 is advanced beyond the distal end of the scope tube 24 and scope channel 22, also called slide, as is shown in FIGS. 10A-10F. It is appreciated that in certain alternate implementations, and as shown in the implementations of FIGS. 11A-11C, the flexion cartridge 50 is capable of being urged beyond the distal end of the rotating tube 20 for flexion, also called nudge. It is further appreciated that the mechanisms of flexion can be used in either implementation.

Flexion of the cartridge 50 in response to the retraction of the actuation cable 56 is shown in FIGS. 11A-C, according to one implementation where the cartridge is advanced beyond the distal end 16 of the rotating tube 20. As shown in this implementation, the cartridge 50 comprises a flexion mechanism 58 further comprising a plurality of internal articulation or flexion units 60A, 60B, 60C, 60D disposed within the cartridge 50. In various implementations, the flexion units 60A, 60B, 60C, 60D are arranged adjacently, and are shaped so as to move as described herein. That is, the units 60A, 60B, 60C, 60D are positioned so as to be capable of articulating movement in response to the retraction of the flexion cable 56.

In these implementations, the flexion cable 56 is in a direct connection with the most distal unit 60A at the unit’s 60A central apex 62A, such that retraction of the cable 56 urges the central apex 60A proximally, as would be understood. In the implementation of FIGS. 11A-C, the units 60A, 60B, 60C, 60D are substantially triangular and configured such that the base apices (shown generally at 64) are adjoining, such that the urging of the central apex 62A of the most distal unit 60A causes the various distal apices 62A, 62B, 62C, 62D cascade into proximity with one another so as to flexion the cartridge 50 and, correspondingly, the laser 42 toward the center of the surgical theater. It is appreciated that while four triangular or pyramidal units 60A, 60B, 60C, 60D are depicted in this implementation as being utilized, other shapes and numbers are of course possible.

Also, it is understood that in this explanatory implementation, a single row of units 60A, 60B, 60C, 60D is utilized, but in practice, several rows of units can be utilized (as shown in FIGS. 12A-E), and in those implementations, the cable 56 may run between the most distal of the units 60A-1, 60A-2 on either side of the cartridge, such as in a U-shape, rather than being anchored as was depicted for simplicity in the implementation of FIGS. 11A-11C. That is, the unit 60A, 60B, 60C, 60D and cable 56 interaction in the cartridge 50 can be configured in several ways to achieve the desired flexion, as would be appreciated by the skilled artisan.

FIG. 12 depicts a distal end view of the device 10 comprising a cartridge 50 disposed in the rotatable tube 20 adjacent to the scope tube and telescope 26, according to one implementation. In this implementation, flexion units 60A-1, 60A-2 are disposed on either side of the laser lumen 15 / laser 42 and connected via the cable 56 for flexion, as described above. That is, the cable 56 extends through the units 60 on either side, optionally anchored at the distal units 60A-1, 60A-2 or free to slide through the units, but in any case able to urge the units 60A-1, 60A-2 to articulate, as described above in relation to FIGS. 11A-C.

Continuing with the implementation of FIG. 12, the cartridge 50 further defines a fluidic lumen 70 around the laser lumen 15 that can accommodate the removal of any smoke or other material generated by the surgical procedure. It is appreciated that in these implementations, the rotatable tube 20 is vented or is in fluidic communication with suction so as to remove the smoke or other material from the surgical theater.

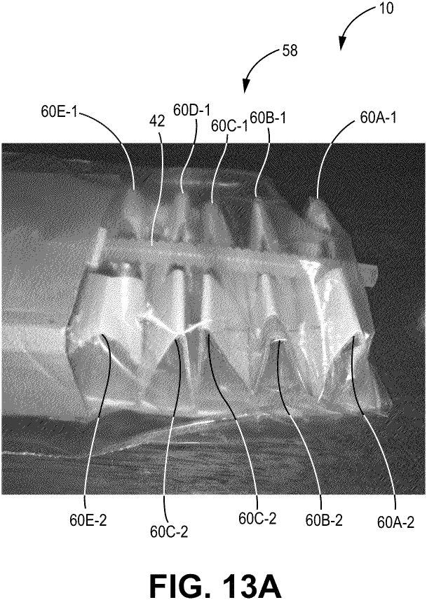

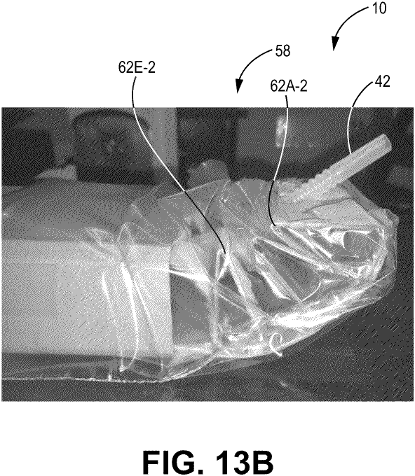

As shown in FIGS. 13A-13B, in various implementations the flexion mechanism 58 consists of a plurality of units 60A-1, 60B-1, 60C-1, 60D-1, 60E-1, 60A-2, 60B-2, 60C-2, 60D-2, 60E-2, disposed on either side of the laser 42 or other tool and in operable communication with the cable 56, as described above. As shown in FIGS. 13A-13B, retraction of the cable 56 results in the articulation of the flexion mechanism by bringing the appropriate apices (represented at 62A-2, 62E-2) into closer proximity and urging the laser 42 toward the center of the surgical theater. While this is one implementation, further implementations are of course possible.

Although the disclosure has been described with reference to certain embodiments, persons skilled in the art will recognize that changes may be made in form and detail without departing from the spirit and scope of the disclosed apparatus, systems and methods.

What is claimed is:

1. An endoscopic device comprising an elongate body comprising:
a) a rotating tube comprising a distal rotating tube end and a proximal rotating tube end; and
b) a scope tube, the scope tube comprising a distal scope tube end and a proximal scope tube end,
wherein the rotating tube is configured for the introduction of a surgical tool and / or suction to the distal rotating tube end for colocalization with a telescope introduced through the scope tube.

2. The device of claim 1, wherein the rotating tube is configured to accommodate a laser.

3. The device of claim 2, wherein the rotating tube is in rotational communication with the scope tube.

4. The device of claim 2, wherein the rotating tube comprises a flexion cartridge disposed therein.

5. The device of claim 4, wherein the flexion cartridge comprises a plurality of flexion units configured to articulate the cartridge.

6. The device of claim 4, wherein the flexion cartridge is configured to be capable of advancement beyond the distal end of the rotating tube.

7. The device of claim 1, wherein the rotating tube is configured to provide suction.

8. An endoscopic device comprising:
a) a rotating tube defined in the lumen, the rotating tube comprising a distal rotating tube end and a proximal rotating tube end;
b) a scope tube, the scope tube comprising a distal scope tube end and a proximal scope tube end; and
c) a flexion cartridge disposed within the rotating tube.

9. The device of claim 8, wherein the rotating tube is in rotational communication with the scope tube.

10. The device of claim 8, wherein the flexion cartridge comprises a plurality of triangular flexion units configured to articulate the cartridge.

11. The device of claim 8, wherein the flexion cartridge is configured to be capable of advancement beyond the distal end of the rotating tube.

12. The device of claim 8, wherein the flexion cartridge is in operational communication with at least one actuator button.

13. The device of claim 8, wherein the flexion cartridge is in operational communication with at least one cable.

14. The device of claim 8, further comprising a locking mechanism.

15. An endoscopic device comprising:
a) a substantially rigid sheath comprising a lumen and having a distal sheath end and proximal sheath end;
b) a rotating tube defined in the lumen, the rotating tube comprising a distal rotating tube end and a proximal rotating tube end;
c) a scope tube defined in the elongate tube opposite the rotating tube, the scope tube comprising a distal scope tube end and a proximal scope tube end;
d) a semi-rigid flexion cartridge disposed at the rotating tube distal end; and
e) at least one locking mechanism.

16. The endoscopic device of claim 15, wherein the rotating tube is configured to rotate around the scope tube.

17. The endoscopic device of claim 15, wherein the flexion cartridge is configured to extending beyond the distal end of the scope tube.

18. The endoscopic device of claim 15, wherein the flexion cartridge is configured to extend beyond the distal end of the rotating tube.

19. The endoscopic device of claim 15, wherein the flexion cartridge comprises a laser lumen.

20. The endoscopic device of claim 15, wherein the flexion cartridge comprises a fluidic lumen.

21. The endoscopic device of claim 15, further comprising at least one actuation button configured to advance the flexion cartridge distally beyond the distal end of the scope tube.

22. The endoscopic device of claim 21, wherein the flexion cartridge is advanced by nudge.

23. The endoscopic device of claim 21, wherein the flexion cartridge is advanced by slide.

24. The endoscopic device of claim 15, wherein the rotating tube is configured to house and rotate a tool in the surgical theater.

25. The endoscopic device of claim 24, wherein the tool is selected from the group consisting of a plasma tool, a coblation tool, a radiofrequency tool, a monopolar cautery tool, a bipolar cautery tool and a needle.

26. The endoscopic device of claim 15, wherein the flexion cartridge comprises a laser lumen.
